(19) United States
(12) Patent Application Publication
(10) Pub. No.: US 2004/0152756 A1
(43) Pub. Date: Aug. 5, 2004

(54) Carvedilol polymorph

(76) Inventors: Wei Chen, Woodbury, MN (US); Marc Galop, Collegeville, PA (US); Choon K. Oh, Collegeville, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/483,217

(22) PCT Filed: Jul. 15, 2002
(86) PCT No.: PCT/US02/22374

(51) Int. Cl. C07D 209/82; A61K 31/403

(52) U.S. Cl. 514/411; 548/444

(57) ABSTRACT

This invention relates to a novel crystalline from of Carvedilol, and to the use of pharmaceutical compositions thereof.

### FIELD OF THE INVENTION

This invention relates to a novel crystalline form of Carvedilol.

### BACKGROUND OF THE INVENTION

The capacity to occur in different crystal structures is known as polymorphism and is known to occur in many organic compounds. These different crystalline forms are known as “polymorphic modifications” or “polymorphs” and are realized in their crystalline state. While polymorphic modifications have the same chemical composition, they differ in packing, geometrical arrangement, and other descriptive properties of the crystalline solid state. As such, these modifications may have different solid-state physical properties such as shape, color density, hardness, deformability, stability, and dissolution properties, etc. Polymorphism of an organic drug molecule and its consequences will be appreciated by the skilled artisan.

Carvedilol is 1-(carbazol-4-yloxy-3-[[2-(o-methoxyphenoxy)ethyl]amino]-2-propanol, and has the following structural formula:

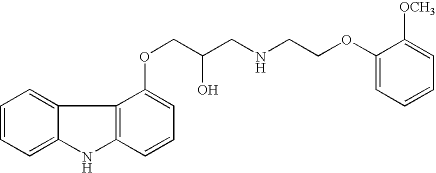

Carvedilol is the subject of U.S. Pat. No. 4,503,067 (the '067 patent), issued Mar. 5, 1985, whose disclosure is incorporated herein by reference in its entirety.

Carvedilol is useful in the treatment of hypertension, congestive heart failure and angina.

According to the instant invention, it has been found that carvedilol exists in a novel crystalline form.

### SUMMARY OF THE INVENTION

This invention relates to a novel crystalline form of carvedilol which is useful in the treatment of hypertension, congestive heart failure and angina.

### BRIEF DESCRIPTION OF THE FIGURES

FIG. 1 is a MDSC thermogram of carvedilol spray-dried powder (Batch N00189).

FIG. 2 is a MDSC thermogram of carvedilol spray-dried powder (Batch N00190).

FIG. 3 is a MDSC thermogram of carvedilol spray-dried powder (Batch N00191).

FIG. 4 are XRPD patterns for batches of carvedilol spray-dried powder and the reference standard of carvedilol drug substance (Form II).

FIG. 5 are FT-IR for batches of carvedilol spray-dried powder and the reference standard of carvedilol drug substance (Form II).

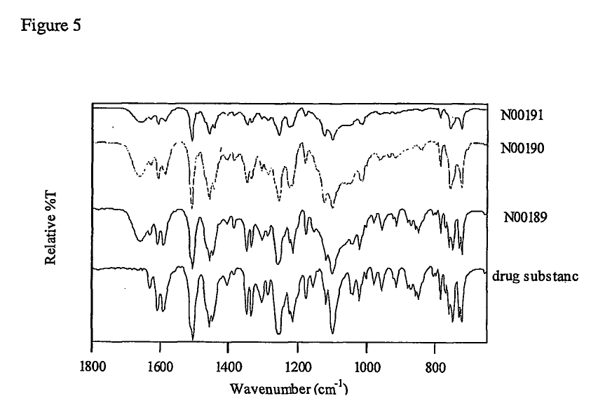

FIG. 6 is a MDSC thermogram of carvedilol drug substance (Form II).

FIG. 7 is a MDSC thermogram of carvedilol Form m.

FIG. 8a is the FT-IR spectra (400-1200 cm⁻1 region) of carvedilol Form II and Form ImI.

FIG. 8b is the FT-IR spectra (2500-4000 cm⁻1 region) of carvedilol Form II and Form m.

FIG. 9 are the XRPD patterns for carvedilol Form III (Lot 46233-138), Form II and carvedilol spray-dried powder (Lot N00191).

FIG. 10 is the ¹³C NQS-edited CP-TOSS spectra for carvedilol Form II and Form mi.

FIG. 11 is a comparison of ¹⁵N CP-MAS spectra for carvedilol Form II and Form 111.

### DETAILED DESCRIPTION OF THE INVENTION

It has now been discovered that Carvedilol can exist as a novel crystalline form (a novel polymorphic form) which differs from previously known forms in its stability, physical properties and spectral data. This novel form is described in this application and is herein referred to as Form III.

This invention also relates to a pharmaceutical composition comprising an effective amount of Form mH with any of the characteristics noted herein, and a pharmaceutically acceptable carrier or diluent thereof.

This invention further relates to the use for treatment of hypertension, congestive heart failure and angina in a mammal in need thereof, which method comprises administering to said mammal an effective amount of Form III with any of the characteristics noted herein.

This invention results from a determination that certain batches of Carvedilol showed differences in their IR spectra, DSC and X-ray powder diffraction pattern. The novel crystalline form of carvedilol of the instant invention may be produced during the wet-bead milling process of Carvedilol Form II, the current commercial form of Carvedilol. The manufacturing process used to prepare the novel polymorphic form of carvedilol included the following steps: 1) preparing Carvedilol suspension which contained 30% drug and 6% Pluronic® F127, 2) wet-bead milling of the Carvedilol suspension, 3) mixing the milled Carvedilol suspension with HEC/PVP solution, and 4) spray drying of the final suspension. The carvediol spray-dried powder produced during this manufacturing process contained 66.7% drug, 13.3% Pluronic® F127, 6.7% Poly(Vinyl Pyrrolidone) (PVP) K30, 13.3% Hydroxyethylcellulose (HEC).

Carvedilol Form III may also be prepared by stirring Carvedilol Form II in water at about 60° C. This novel crystalline form melts at about 92-95° C., while Form II melts at about 1141115° C.

Experimental Details:

The procedure to prepare the new crystalline form is summarized below:

1. Add water into a glass beaker (or flask), and stir vigorously using a magnetic stir bar. Slowly add Carvedilol Form II powder into water. The Carvedilol to water ratio used has been between 1:200 and 1:40 gram/ml.

2. Heat the suspension to 60-65° C. Keep stirring at this temperature with the container being covered by aluminum foil.

3. Monitor the conversion process by analyzing the suspension using DSC as described below. Usually it takes 2 to 3 days for the conversion to complete.

4. Separate the solids from liquid by filtration when the conversion is complete. Wash the solids with water a couple of times. Subsequently dry the obtained solids in a desiccator under vacuum, or at 60 IC under vacuum.

Methods

Differential Scanning Calorimetry (DSC):

DSC measurements were performed with a MDSC 2920 (IA Instruments, Inc.). About 5 mg of the powder was placed in an open aluminum pan. The sample was scanned at 10° C./min from 0° C. to 160° C. The suspension sample was placed in a plastic weighing dish and excess liquid was removed manually by paper tissues. The solids were then dried in a fume hood before DSC measurements.

Fourier Transform Infrared Spectroscopy (FI-IR):

Approximately 2 mg of sample was diluted with 300 mg of dried potassium bromide (KBr). The mixture was ground with a mortar and pestle, then transferred to a die that is placed under high pressure for 3 minutes. The instrument was a PerkinElmer Spectrum GX FTIR instrument. Ten scans were collected at 4 cm⁻¹ resolution.

X-Ray Powder Diffraction (XRPD):

XRPD patterns were collected using a Bruker D8 Advance X-ray Diffractometer. Approximately 60 mg of sample was gently flattened on a silicon sample holder and scanned from 2 to 35 degrees two-theta, at 0.02 degrees two-theta per step and a step time of 2.5 seconds. The suspension samples were simply placed on the sample holder. The sample was rotated at 25 rpm.

Solid State Nuclear Magnetic Resonance (Solid-State NMR):

Approximately 500 mg of each sample was packed into 7-mm magic-angle spinning rotors and spun at 5 kHz. The NQS (non-quaternary suppression) edited CP-MAS pulse sequence (cross-polarization with magic angle spinning) was used to measure the ¹⁵N spectrum of the samples. The ¹³C spectrum of each sample was recorded using a CP-TOSS pulse sequence (cross polarization with total suppression of sidebands). A Brucker AMX/ARX-360 spectrometer was used for this work.

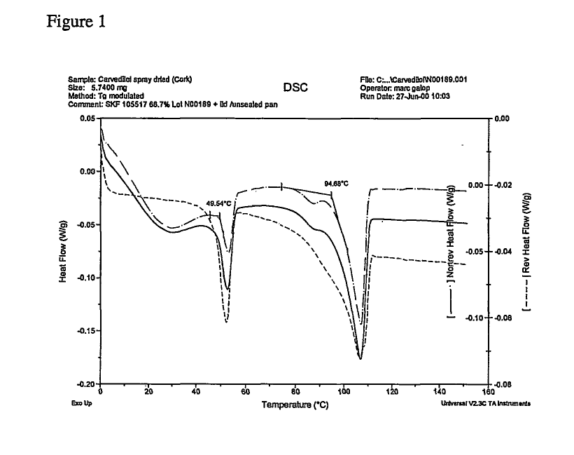

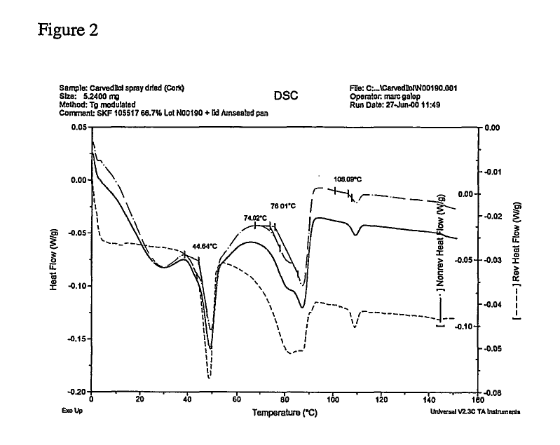

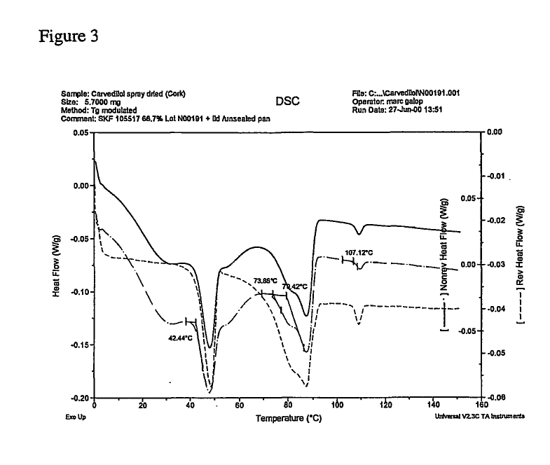

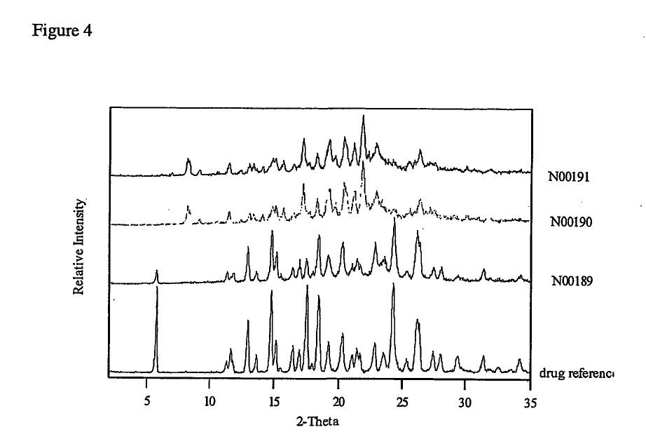

FIGS. 1 to 3 show the MDSC thermograms of three batches (N00189, N00190 and N00191) of Carvedilol spray-dried powder. FIG. 4 is the XRPD patterns for the batches of Carvedilol spray-dried powder together with the reference standard of Carvedilol drug substance.

There were two major endothermic peaks on the thermogram of N00189, near 50° C. and 90° C., corresponding to the melting of Pluronic F127 and the drug, respectively. It should be noted that the melting onset temperature of the drug was much lower than the pure drug (113-115° C. for Carvedilol Form II). This was due to the interactions between the drug particles and Pluronic F127 adsorbed on the surface of the drug particles. Both XRPD pattern and FT-IR spectrum for N00189 demonstrated that Carvedilol existed as Form II in the spray-dried powder, same as the reference standard of the drug substance.

Batches N00190 and N00191 spray-dried powder were different from batch N00189. Indeed, a new endothermic peak appeared near 75° C. on the thermograms of these two batches. This endothermic peak corresponded to the melting of a distinct and new crystalline form. This new crystalline form exhibited very different XRPD patterns and FT-IR spectra from the Form II drug substance.

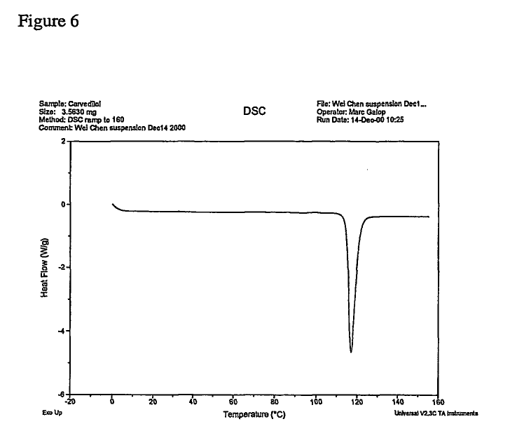

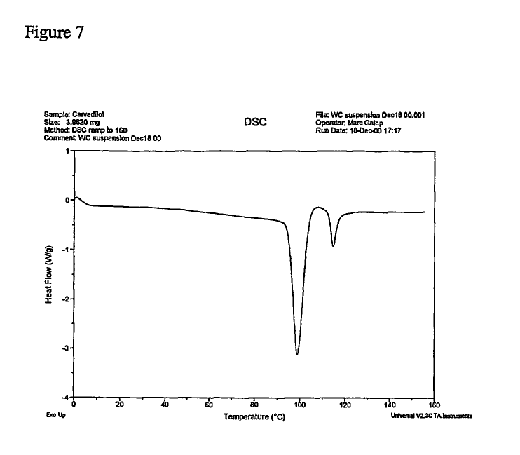

FIGS. 6 and 7 show the typical thermograms of Carvedilol Form II and Form II, respectively.

As shown in FIG. 7, the thermogram of the new material exhibited a major endotherm (melting event) near 92° C., immediately followed by a small exotherm (recrystallization), and a small endotherm near 110° C. One possible explanation for this behavior is that, after the new crystalline form melted, a small amount of the melt recrystallized into Carvedilol Form II and then gave the second melting peak.

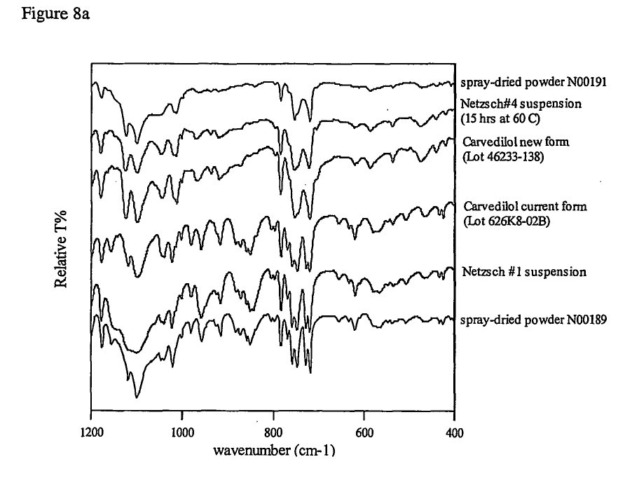

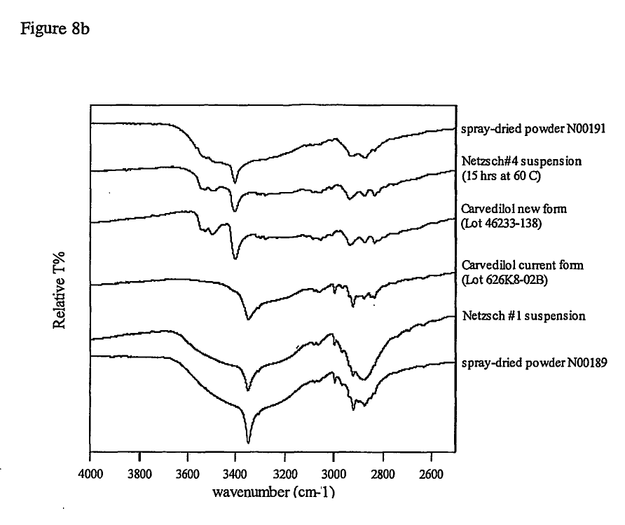

FIGS. 8a and 8b show a comparison of FT-IR spectra for the new form and the currently used form (Form II) in drug substance, milled suspensions and spray-dried powders. It can be seen that the drug exists as Form II in Netzsch#1 milled suspension and spray-dried powder N011 89. Most importantly, Form I (Lot 46233-138) exhibited the same FT-IR spectral features as the new form discovered in spray-dried powder N00191 and heat-treated Netzsch#4 suspension. Therefore, the new form material prepared by stirring Carvedilol Form II suspension is the same crystalline form as the one obtained in the two batches of Carvedilol spray-dried powder.

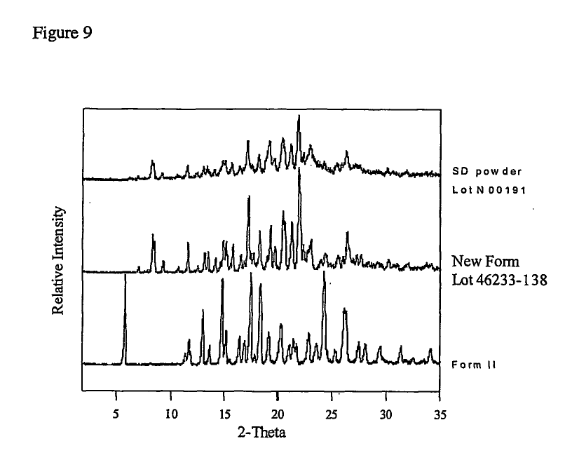

The XRPD pattern for the new form material is displayed in FIG. 9 together with the currently used form of Carvedilol (Form U1) and Carvedilol spray-dried powder Lot N00191. Again, the diffraction pattern from the new form material matched that from the spray-dried powder Lot N00191, indicating the same crystalline structure. Carvedilol Form III has an X-ray powder diffraction pattern which comprises characteristic peaks at about 8.4, 17.4 and 22.0 degrees two-theta.

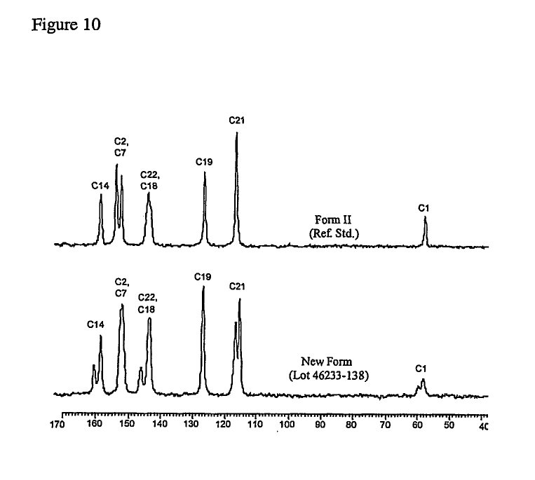

The ¹³C NQS-edited CP-TOSS spectra for the Carvedilol Form III and Form II are illustrated in FIG. 10, where only quaternary and methyl resonances are present. The assignments of each peak were made, in reference to the structure of Carvedilol (Scheme 1).

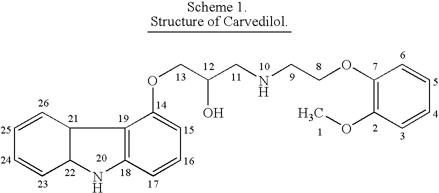

From its NMR spectrum, it appears that Form II sample contains one molecule per crystalline unit cell. However, the typical splitting of peaks observed in compounds with more than one molecule per unit cell is observed for the Carvedilol new form. The splitting effect could also arise from a mixture of two or more phases; however, since the spectra do not coincide, these phases must also be new.

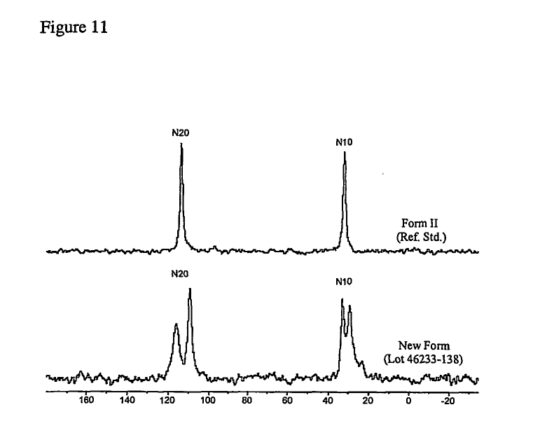

The ¹⁵N CP-MAS spectra of the two forms are shown in FIG. 11. Again, Form II appears to contain a single molecule per unit cell, while the Form m contains multiple resonances for each type of nitrogen, consistent with either multiple molecules per unit cell or multiple new phases.

The above data demonstrates that a new crystalline form of Carvedilol (Form III) is prepared from Carvedilol Form II.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the are can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

What is claimed is:

1. A compound which is carvedilol Form m.

2. The compound according to claim 1 having an X-ray powder diffraction pattern which comprises characteristic peaks as expressed in FIG. 9, Lot 46233-138.

3. The compound according to claim 1 having an X-ray powder diffraction pattern which comprises characteristic peaks at about 8.4, 17.4 and 22.0 degrees two-theta.

4. The compound according to claim 1 having an infrared spectrum which comprises characteristic absorption bands expressed in reciprocal centimeters as described in FIGS. 8a and 8b, Lot 46233-138.

5. The compound according to claim 1 having a DSC enothermic peak at about 92° C.

6. The compound according to claim 1 having a melting point at about 92-95° C.

7. A pharmaceutical composition comprising the compound according to any one of claims 1 to 6, and a pharmaceutically acceptable carrier or diluent.

8. A method of treating hypertension, angina or congestive heart failure in a mammal in need thereof, which method comprises administering to said mammal an effective amount of the compound according to any one of claims 1 to 6.
